(19) United States
(12) Patent Application Publication
(10) Pub. No.: US 2018/0292381 A1
(43) Pub. Date: Oct. 11, 2018

(54) HEALTH DIAGNOSTIC WASTE CONTAINER

(71) Applicant: Pixie Scientific, LLC, New York, NY (US)

(72) Inventors: Yaroslav FAYBISHENKO, New York, NY (US); Neer ASHERIE, New York, NY (US)

(73) Assignee: Pixie Scientific, LLC, New York, NY (US)

(21) Appl. No.: 15/948,961

(22) Filed: Apr. 9, 2018

Related U.S. Application Data
(60) Provisional application No. 62/483,188, filed on Apr. 7, 2017.

(51) Int. Cl. G01N 33/487; B65F 1/16; B65F 1/06; G01N 21/78; G01N 21/359; G01N 5/00; G08B 21/02; G08B 5/22

(52) U.S. Cl. CPC G01N 33/48778; B65F 1/1646; B65F 1/06; G01N 21/78; G01N 21/359; B65F 2210/168; G08B 21/02; G08B 5/22; B65F 2240/132; B65F 2210/184; B65F 2210/128; G01N 5/00

(57) ABSTRACT

Waste container systems including a canister that defines an interior space configured to receive and store diapers that include bodily waste, a base configured to support the canister, and a cover secured to the top of the canister. The waste container system includes one or more sensors configured to detect a characteristic of the bodily waste deposited into the canister and transmit the detected characteristic to a processor configured to correlate the detected characteristics with a health condition.

### CROSS-REFERENCE TO RELATED APPLICATIONS

The present application claims the benefit of priority under 35 U.S.C. § 119(e) of U.S. Provisional Patent Application Ser. No. 62/483,188 for a “HEALTH DIAGNOSTIC WASTE CONTAINER”, filed Apr. 7, 2017, hereby incorporated by reference for all purposes.

### BACKGROUND

A variety of illnesses or health conditions may be detected and/or diagnosed by analysis of bodily waste, such as urine and stool. Although laboratory tests are readily available, the inconvenience and expense of such testing prohibits their routine use in health monitoring.

What is needed is a system for easily monitoring bodily waste in an ongoing manner, in order evaluate characteristics such as hydration level, kidney function, impending infections, and other potential metabolic and endocrine disease states.

### SUMMARY

The present disclosure is directed to a waste container system including a canister that defines an interior space configured to receive and store diapers that include bodily waste, a base configured to support the canister, and a cover secured to the top of the canister. The waste container system includes one or more sensors configured to detect a characteristic of the bodily waste deposited into the canister and transmit the detected characteristic to a processor configured to correlate the detected characteristics with a health condition.

### BRIEF DESCRIPTION OF THE DRAWINGS

FIG. 1 depicts an exemplary waste container system according to the present disclosure.

FIG. 2 depicts a partially exploded view of the exemplary waste container system of FIG. 1.

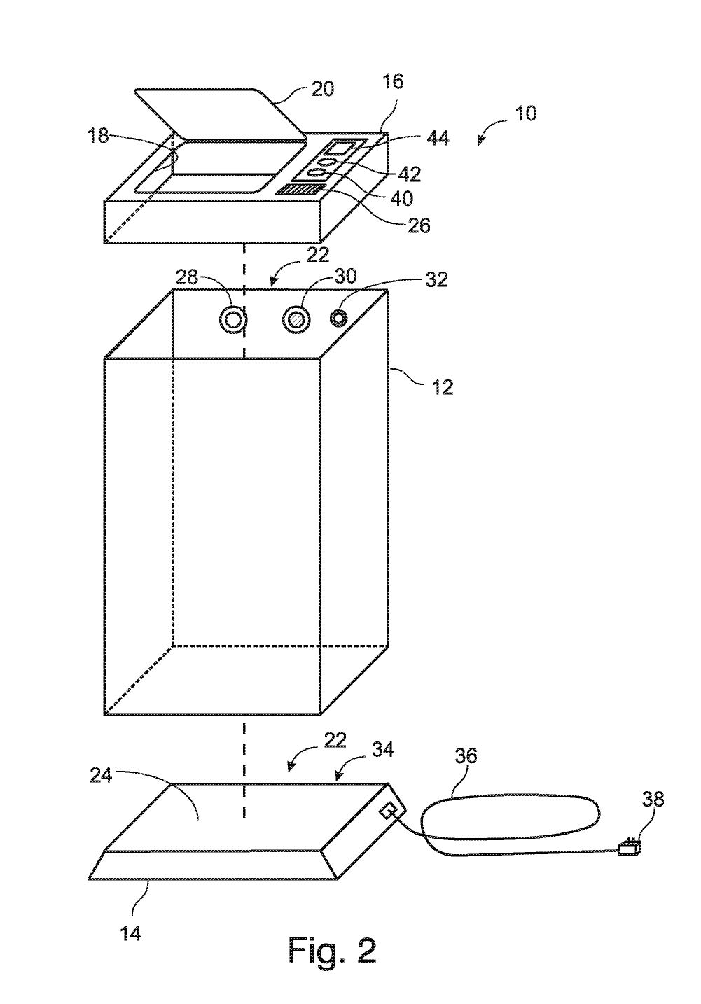

### DETAILED DESCRIPTION

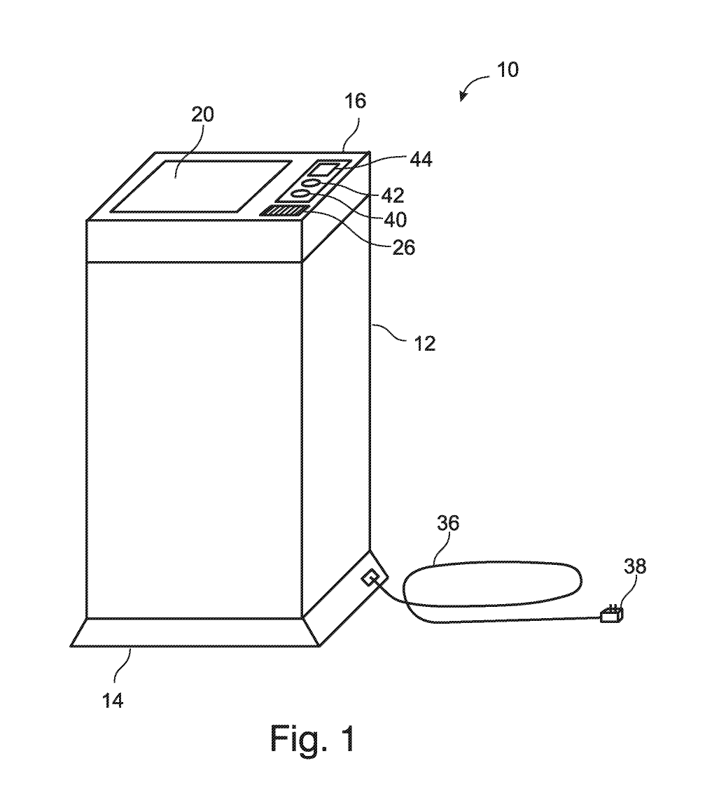

A waste container system 10 is depicted semi-schematically in FIG. 1. The waste container 10 may be configured to receive bodily wastes, to detect one or more characteristics of the received bodily wastes using one or more sensors, and to analyze the detected characteristics to correlate the detected characteristics with one or more health conditions. Although waste container 10 may receive bodily wastes in any of a variety of forms, it may be most convenient to receive bodily wastes associated with an infant or adult diaper, or a cleansing wipe. For this reason, the waste container system 10 may be used in place of a traditional diaper pail, for storing used diapers until they can be disposed of. Where the present disclosure refers to a diaper, it should be appreciated that the present disclosure is not limited to the use of diapers, and that any other compatible vehicle for the addition of bodily waste to the waste container system that permits the subsequent detection of one or more characteristics of the bodily wastes may be a suitable vehicle.

Waste container system 10 may include a canister 12 that defines an interior storage space for receiving diapers (not shown). Canister 12 may be equipped with a base 14 that supports the canister, and a cover 16 that covers the top of canister 12. When assembled, canister 12, base 14, and cover 16 form a diaper storage unit that may be capable of storing multiple soiled diapers with minimal odor until their further disposal. Canister 12 may be configured to receive a canister liner, such as a disposable plastic bag or a nested inner container, in order to simplify the storage and removal of soiled diapers. For the purposes of this disclosure the liner is not shown.

Cover 16 may rest on top of canister 12. Cover 16 may be held in place simply by gravity, and may be configured to rest on an interior shelf or lip around the mouth of canister 12. Alternatively or in addition, cover 16 may be secured to canister 12 by any suitable mechanism, such as one or more latches, straps, interlocks, or other conventional retention mechanisms. In one aspect of the disclosure, cover 16 is secured to canister 12 by a child-safe lock or latch. Cover 16 may be configured to be removable so that soiled diapers (optionally within a bag or liner) may be removed for further disposal.

Cover 16 may define an opening 18 through which diapers may be added to canister 12. Opening 18 may be sealed by an openable lid 20, which may be attached to cover 16 via one or more hinges, or may simply rest atop cover 16 or on a circumferential lip around opening 18. Where openable lid 20 is attached to cover 16 via one or more hinges, lid 20 may be urged to remain in a closed position via a biasing mechanism, for example one or more springs.

Base 14 may be configured to serve as a secure platform for canister 12, and may help retain canister 12 in an upright orientation. Base 14 may be configured to resist the tipping of canister 12 for example by including a depressed area configured to receive canister 12, or by incorporating a magnet configured to interact with a complementary magnet within canister 12, among other mechanisms to resist tipping. Canister 12 may simply rest on top of base 14, or may be engaged to base 14 via one or more mechanisms such as latches, straps, interlocks, and the like.

Waste container system 10 may include one or more sensors 22 that are configured to detect a characteristic of waste deposited in waste container system 10. The sensors may be configured to detect a plurality of different characteristics, or different types of characteristics.

In one aspect of the disclosure, a characteristic being detected is a weight of the bodily waste being added to waste container system 10, and the sensor may correspond to a weight-detection system 24, such as an analytical balance or force-sensitive resistor, incorporated in waste container system 10. The weight-detection system 24 may be incorporated in the bottom surface of canister 12, or incorporated in base 14.

Alternatively or in addition, the waste container system 10 may include sensors 22 that may be configured to detect characteristics based on reflected electromagnetic radiation. For example, the cover 16 may include a scanner 26 that is configured to capture an image of a diaper, particularly where the diaper may include one or more computer-readable indicia. Scanner 26 may be used to scan barcodes, QR codes, or any other means of encoding digital information that may be present on either the diaper, or the diaper packaging in order to identify the diaper being used. In doing so, information about the diaper, such as the type of diaper, the clean weight of the diaper, or the types of indicating regions present on the diaper, may be detected and recorded. The scanner may further be configured to capture an image of an object, in order to subject it to additional analysis, for example by computer vision techniques or other image-analyzing algorithms.

In those cases where scanner 26 scans a computer-readable indication that the diaper being placed inside canister 12 includes one or more colorimetric indicators, the indication may then trigger the activation of one or more cameras 28 either inside or outside canister 12. The cameras may be sensitive to light in the visual region, and so may be capable of capturing accurate color information from a colorimetric indicator, which may then be correlated with one or more characteristics of the diaper or its contents.

For example, one or more colorimetric indicators may be may be configured to produce a visual indication of one or more analytes contained in the bodily waste present in the diaper. For example, one or more colorimetric indicators may include one or more reagents configured to react with one or more specific analytes which may be contained in the waste to produce a visual indication (e.g., a change in color or color intensity) that communicates a qualitative or quantitative indication of a presence of, or an amount of the one or more analytes contained in the sample.

The colorimetric indicators may be selected to, for example, indicate the presence of ketones in urine, a specific gravity of urine, the presence of glucose, bilirubin, blood, pH, protein, urobilinogen, nitrite, leukocytes, and/or creatinine, among others. A variety of chemical strategies and methods of detecting analytes by producing a visual indication are well known in the art. For example, see U.S. Pat. Nos. 9,131,893; 5,516,700; 4,318,709 4,147,514; and 3,146,070 and U.S. Patent Appl. Publ. 2016/0338883 which are all hereby incorporated by reference for all purposes.

In order to obtain the colorimetric data displayed by such colorimetric indicators, the canister 12 may include one or more devices sensitive to various selected wavelengths of electromagnetic radiation. In one aspect, canister 12 may include one or more cameras 28 that are sensitive to the visible region of the spectrum. In one aspect of the disclosure, at least two cameras 28 may be mounted on the front and back inner walls of canister 12.

Alternatively, or in addition, waste container system 10 may include one or more electromagnetic detectors 30 that may be sensitive to near-infrared wavelengths (750-2400 nm). The near-infrared detector 30 may be used, but is not required to be used, in conjunction with a near-infrared illumination source 32, which may be disposed on the underside of cover 16, for example, or on an inner wall of canister 12. The near-infrared detector 30 may be used to measure the near-infrared reflectance of stool that may be present in the bodily waste deposited in the waste container system 10. Such a near-infrared detector may be mounted on an inner wall of canister 12, for example adjacent to cameras 28.

Alternatively, or in addition, waste container system 10 may include one or more electromagnetic detectors 30 that may be configured to be sensitive to visible/ultraviolet wavelengths. A visible/ultraviolet reflectance detector, for example, may detect an amount of light reflected in the visible and ultraviolet spectrum (220-750 nm), and could be used to measure light emitted from one or more fluorescent sensors associated with a diaper placed within waste container system 10. Such sensors might, for example, be activated by specific chemical reactions designed to detect and/or quantitate a target analyte in the diaper. Where the detector 30 is a visible/ultraviolet reflectance detector, the detector may be mounted on an inner wall of canister 12, for example adjacent to or at the same level as cameras 28.

As used herein, a camera or detector may include one or more lenses or other optics to direct and/or focus light of the desired wavelength. The camera or detector may employ a CCD device to image the light, or any other suitable detection array or device.

Base 14 may additionally include a processor 34, where processor 34 is configured to acquire, transmit, process, analyze, and/or store the data received by any sensor 22 included in waste container system 10, for example such as receiving scanned images from scanner 26, visual data from cameras 28, near-infrared reflectance data from detectors 30, and/or weight information recorded by weight-detection system 24. Processor 34 may receive such data from canister 12 via any suitable communication network, including a hard-wired communication network. Alternatively or in addition, processor 34 may receive such data from canister 12 via a wireless communication network.

Processor 34 may be configured to process and/or analyze the data collected by sensors 22. For example, the collected data may be analyzed in order to evaluate whether any of the results may indicate, or correlate with, a given health condition. A health condition may be a normal health condition, or an abnormal health condition. Processor 34 may be configured to determine one or more at least quasi-quantitative and/or qualitative indications of one or more analytes contained in the bodily waste placed in waste container system 10. For example, processor 34 may be used to determine the fat and nitrogen content of a sample of stool, by comparing the near-infrared reflectance data captured by detector 30 with known calibration curves, among other analytes and analytical methodologies.

When processor 34 determines one or more indications based on collected data, the indications may be displayed on display 44, or transmitted to an external device. Alternatively or in addition, the data collected by sensors 22 may be transmitted to an external device for processing in order to determine if any of the results indicate a given health condition.

When processor 34 is in communication with an external device, the device may be a networked processor or a mobile device, and the communication may occur via a wireless data connection, or via local- or large-area network (such as the Internet). Base 14 may therefore include the necessary hardware to conduct WiFi communication and/or Bluetooth communication with another device. Alternatively or in addition, waste container system 10 may include the necessary hardware to engage in Near Field Communication (NFC) with another device that may be incorporated, for example, in or on cover 16. In one aspect of the disclosed waste container system, the NFC hardware may be incorporated in or utilize scanner 26. Where the electronics employed by waste container system 10 may require data ports, such data ports may be disposed on, for example, the rear of base 14.

Base 14 may additionally and optionally include a power supply for the devices incorporated in waste container system 10, for example by including a power connection 36 for an AC adapter 38 configured to be plugged into a standard electrical socket, as shown in FIG. 1.

The upper surface of cover 16 may additionally include a number of features to facilitate the user operation of waste container system 10. For example, and without limitation, cover 16 may include a power switch 40, a data input system 42 to record characteristics of a given diaper (such as whether it contains stool or urine), and a status display 44 to provide information to a user. Data input system 42 may correspond to a simple mechanical button or toggle, or may incorporate multiple keys or a keyboard for the entry of more complex data.

Status display 44 may simply include lights that are configured to indicate when waste container system 10 is powered and/or functioning properly. Alternatively, status display 44 may include a screen such as an LCD screen configured to give more detailed feedback on waste container system 10 operations. Where status display 44 includes a screen, the screen may be a touchscreen, thereby additionally facilitating user input of information. Alternatively, or in addition, the screen may be used to display a result of the analysis of the bodily waste deposited in the waste container. That is, status display 44 may include a screen that is configured to display an identification of one or more health conditions correlated by processor 34 with data collected by one or more of sensors 22 corresponding to one or more characteristics of the bodily waste. Alternatively, or in addition, the identified health conditions may be transmitted to one or more external devices, such as a user's portable digital device, or a computer connected to the Internet.

Although the present waste container system has been shown and described with reference to the foregoing operational principles and preferred embodiments, it will be apparent to those skilled in the art that various changes in form and detail may be made without departing from the spirit and scope of the disclosure. The present disclosure is intended to embrace all such alternatives, modifications and variances and include all novel and non-obvious combinations and subcombinations of the various elements, features, functions and/or properties disclosed herein. Various combinations and subcombinations of features, functions, elements, and/or properties may be claimed through presentation of a related application.

What is claimed is:

1. A waste container system, comprising:
a canister defining an interior space configured to receive and store diapers that include bodily waste;
a base configured to support the canister;
a cover secured to a top of the canister;
wherein the waste container system includes one or more sensors configured to detect a characteristic of the bodily waste; and transmit the detected characteristic to a processor configured to correlate the detected characteristics with a health condition.

2. The waste container system of claim 1, wherein the cover defines an opening for receiving the diapers, and includes an openable lid for sealing the opening.

3. The waste container system of claim 1, wherein the one or more sensors include one or more of a weight detection system, a scanner, a visible wavelength camera, a near-infrared reflectance detector, or a visible/ultraviolet reflectance detector.

4. The waste container system of claim 1, wherein the processor is configured to receive the detected characteristic from the canister via wireless communication.

5. The waste container system of claim 1, wherein the processor is disposed in the base of the waste container system.

6. The waste container system of claim 1, further comprising a wired or wireless communication system for communicating with a networked processor or mobile device.

7. The waste container system of claim 6, wherein the communication system includes one or more of a WiFi, Bluetooth, and Near Field Communication system, and hardware for each communication system is disposed in the base or the cover of the waste container system.

8. The waste container system of claim 1, wherein the cover includes one or more of a power switch, a data input system, and a status display.

9. The waste container system of claim 1, further comprising one or more canister liners configured to receive and contain the diapers that include bodily waste.

10. A method of detecting a characteristic of bodily waste, comprising:
depositing a sample of bodily waste into a waste container system, wherein the waste container system includes
a canister defining an interior space configured to receive and store diapers that include bodily waste;
a base configured to support the canister; and
a cover secured to a top of the canister;
wherein the waste container system further includes one or more sensors configured to detect a characteristic of the bodily waste, and a processor configured to correlate the detected characteristic with a health condition;
detecting by the sensors the at least one characteristic of the deposited bodily waste;
transmitting the detected characteristic to the processor; and
correlating by the processor the detected characteristic with the health condition.

11. The method of claim 10, further comprising:
reporting the correlated health condition to a user of the waste container system.

12. The method of claim 11, wherein reporting the correlated health condition to the user includes displaying an identification of the correlated health condition on a status display screen.

13. The method of claim 11, wherein reporting the correlated health condition to the user includes transmitting an identification of the correlated health condition to a user's portable digital device.

14. The method of claim 11, wherein reporting the correlated health condition to the user includes transmitting an identification of the correlated health condition to a computer connected to a local-area network or large-area network.
